(19) United States
(12) Patent Application Publication
(10) Pub. No.: US 2025/0216302 A1
(43) Pub. Date: Jul. 3, 2025

(54) DEVICE FOR PRESSING CASSETTE AND EMBEDDER HAVING SAME

(71) Applicant: Leica Biosystems Nussloch GmbH, Nussloch (DE)

(72) Inventors: Hongjian TAO, Shanghai (CN); Chun JIN, Shanghai (CN); Yangming LV, Shanghai (CN)

(21) Appl. No.: 18/851,019

(22) PCT Filed: Mar. 31, 2022
(86) PCT No.: PCT/CN2022/084519

§ 371 Date: Sep. 25, 2024

(51) Int. Cl. G01N 1/36

(52) U.S. Cl. CPC G01N 1/36; G01N 2001/366

(57) ABSTRACT

A device for pressing a cassette is provided. The cassette includes an outer frame and an inner frame mounted in the outer frame, and the cassette is configured to be held in a groove of a mold. The device includes a pressing member; an abutment member configured to abut against the cassette; a sensor coupled between the pressing member and the abutment member and configured to measure a pressing force on the cassette, and a guide member configured to be mounted on the mold. The pressing member is detachably mounted to the guide member and slidable on the guide member in a vertical direction. The pressing force on the cassette can be detected in real time by the device according to the present disclosure.

### CROSS-REFERENCE TO RELATED APPLICATION(S)

This application is the national phase of PCT Application No. PCT/CN2022/084519 filed on Mar. 31, 2022, the disclosure of which is incorporated herein by reference.

### FIELD

The present disclosure relates to the field of biopsy, and more particularly to a device for pressing a cassette and an embedder having the same.

### BACKGROUND

During embedding process of a tissue sample, the cassette containing the tissue sample is held in a mold to define a space, and liquid paraffin may be dispensed into the space for producing solidified paraffin block embedding the tissue sample. In the related art, the cassette includes an outer frame and an inner frame mounted in the outer frame, and a user typically holds the outer frame by fingers with feelings and experience. During auto-embedding process, there is a risk to destroy the tissue sample in the cassette when the cassette is pressed into the mold, leading to poor quality of resulting paraffin block.

### SUMMARY

Embodiments of the present disclosure seek to solve at least one of the problems existing in the related art to at least some extent. Embodiments of present disclosure provide a device for pressing a cassette, which can measure a pressing force on the cassette in real time, to improve the quality of the resulting paraffin block and improve the stability of the cassette during the embedding process.

Embodiments of the present disclosure provide a device for pressing a cassette. The cassette includes an outer frame and an inner frame mounted in the outer frame, and the cassette is configured to be held in a groove of a mold. The device includes a pressing member; an abutment member configured to abut against the cassette; a sensor coupled between the pressing member and the abutment member and configured to measure a pressing force on the cassette, and a guide member configured to be mounted on the mold. The pressing member is detachably mounted to the guide member and slidable on the guide member in a vertical direction.

With the device according to embodiments of the present disclosure, a user may push the pressing member, and force the abutment member via the sensor to abut against the cassette, the cassette may be held in the groove of the mold, and the pressing force on the cassette may be measured by the sensor coupled between the pressing member and the abutment member in real time.

In some embodiments, the pressing member defines a first hole through the pressing member in the vertical direction, and the guide member is fitted in the first hole. Thus, the pressing member can slide along the guide member.

In some embodiments, the device further includes an elastic member coupled between the pressing member and the mold and configured to force the pressing member towards the outer frame of the cassette. The abutment member configured to abut against the outer frame of the cassette. Thus, the abutment member can be abutted against the outer frame of the cassette under the action of the elastic member.

In some embodiments, the abutment member includes a protrusion extending towards the outer frame of the cassette in the vertical direction. Thus, the protrusion of the abutment member can be abutted against the outer frame of the cassette.

In some embodiments, the abutment member defines a first recess in a side of the abutment member, and the first recess is configured to position a dispensing tube of a paraffin dispensing device.

In some embodiments, the device further includes a rod configured to be detachably mounted on the mold. The pressing member is slidably mounted to the rod, a free end of the rod extends beyond a surface of the pressing member away from the mold, and the elastic member is coupled between the free end of the rod and the surface of the pressing member away from the mold. Thus, the elastic member is coupled to the mold by the rod.

In some embodiments, the pressing member defines a second hole through the pressing member in the vertical direction, and the rod is fitted in the second hole. Thus, the rod will not interfere with the movement of the pressing member along the guide member.

In some embodiments, the device further includes an adjustment screw screwed to the free end of the rod. The elastic member is coupled between the adjustment screw and the surface of the pressing member away from the mold. Thus, the action force of the elastic member can be adjusted by the adjustment screw to adjust the pressing force on the outer frame of the cassette.

In some embodiments, the device further includes an elastic member coupled between the pressing member and the mold and configured to force the pressing member away from the inner frame of the cassette. The abutment member configured to abut against the inner frame of the cassette. Thus, the abutment member can be abutted against the inner frame of the cassette under the action of the elastic member.

In some embodiments, the elastic member is coupled between the mold and a surface of the pressing member facing the mold. Thus, the elastic member may push the pressing member away from the mold.

In some embodiments, the device further includes an adjustment ring fitted over the guide member and arranged between the elastic member and the mold. Thus, the action force of the guide member can be adjusted by the adjustment ring.

In some embodiments, the sensor is elastically coupled to a monitor, and configured to send a signal to the monitor for display of a measure value of the pressing force on the cassette. Thus, the pressing force on the cassette can be read by the user in real time.

In some embodiments, the device further includes a further pressing member; a further abutment member configured to abut against the inner frame of the cassette; a further sensor coupled between the further pressing member and the further abutment member and configured to measure a pressing force on inner frame of the cassette, and a further guide member configured to be mounted on the mold. The further pressing member is detachably mounted to the further guide member and slidable on the further guide member in the vertical direction. Thus, the device can press the outer frame and the inner frame of the cassette at the same time.

In some embodiments, the further pressing member defines a third hole through the further pressing member in the vertical direction, and the further guide member is fitted in the third hole. Thus, the further pressing member can slide along the further guide member.

In some embodiments, the abutment member defines a first slot through the abutment member in the vertical direction, the pressing member defines a second slot through the pressing member in the vertical direction, and the first slot and the second slot are aligned in the vertical direction for passage of the further abutment member. Thus, the abutment member and the pressing member will not interfere with movement of the further abutment member.

In some embodiments, the further abutment member includes a leg extending towards the inner frame of the cassette in the vertical direction. Thus, the leg of the further abutment member can be abutted against the inner frame of the cassette.

In some embodiments, the device further includes a further elastic member coupled between the further pressing member and the further mold and configured to force the pressing member away from the inner frame of the cassette.

In some embodiments, the further elastic member is coupled between the mold and a surface of the further pressing member facing the mold. Thus, the further elastic member may push the further pressing member away from the inner frame of the mold.

In some embodiments, the device further includes an adjustment ring fitted over the further guide member and coupled between the further elastic member and the mold. Thus, the action force of the further guide member can be adjusted by the adjustment ring.

In some embodiments, the further abutment member defines a second recess in a side of the further abutment member, the further pressing member defines a third recess in a side of the further pressing member, and the second recess and the third recess are aligned in the vertical direction to position a dispensing tube of a paraffin dispensing device.

Embodiments of the present disclosure further provide an embedder for automatic embedding tissue samples in paraffin with an integrated device according to any of the above embodiments.

Additional aspects and advantages of embodiments of present disclosure will be given in part in the following descriptions, become apparent in part from the following descriptions, or be learned from the practice of the embodiments of the present disclosure.

### BRIEF DESCRIPTION OF THE DRAWINGS

These and other aspects and advantages of embodiments of the present disclosure will become apparent and more readily appreciated from the following descriptions made with reference to the drawings, in which:

FIG. 1 is a perspective view of a device for pressing a cassette according to an embodiment of the present disclosure.

FIG. 2 is a perspective view of a device for pressing a cassette according to another embodiment of the present disclosure.

FIG. 3 is a perspective view of a device for pressing a cassette according to yet another embodiment of the present disclosure.

### DETAILED DESCRIPTION OF THE DISCLOSURE

Reference will be made in detail to embodiments of the present disclosure. The embodiments described herein with reference to drawings are explanatory, illustrative, and used to generally understand the present disclosure. The embodiments shall not be construed to limit the present disclosure. The same or similar elements and the elements having same or similar functions are denoted by like reference numerals throughout the descriptions.

In the specification, unless specified or limited otherwise, relative terms such as “central”, “longitudinal”, “lateral”, “front”, “rear”, “right”, “left”, “inner”, “outer”, “lower”, “upper”, “horizontal”, “vertical”, “above”, “below”, “up”, “top”, “bottom” as well as derivative thereof (e.g., “horizontally”, “downwardly”, “upwardly”, etc.) should be construed to refer to the orientation as then described or as shown in the drawings under discussion. These relative terms are for convenience of description and do not require that the present disclosure be constructed or operated in a particular orientation.

Terms concerning attachments, coupling and the like, such as “connected” and “interconnected”, refer to a relationship in which structures are secured or attached to one another either directly or indirectly through intervening structures, as well as both movable or rigid attachments or relationships, unless expressly described otherwise.

Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

In the description of the present disclosure, it should be understood that, unless specified or limited otherwise, the terms “mounted,” “connected,” and “coupled” and variations thereof are used broadly and encompass such as mechanical or electrical mountings, connections and couplings, also can be inner mountings, connections and couplings of two components, and further can be direct and indirect mountings, connections, and couplings, which can be understood by those skilled in the art according to the detail embodiment of the present disclosure.

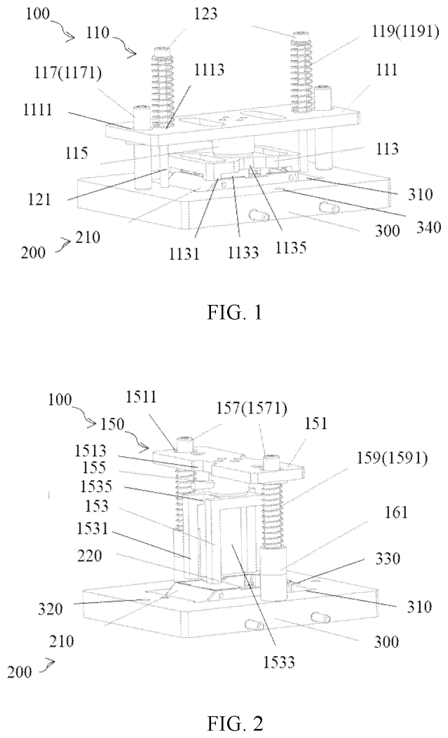

A device 100 for pressing a cassette 200 according to embodiments of the present disclosure will be described below with reference to FIGS. 1 to 3.

The cassette 200 may include an outer frame 210 and an inner frame 220. The inner frame 220 may be mounted in the outer frame 210 to define an accommodating space for receiving a tissue sample. During embedding process, the cassette 200 may be held in a groove 310 of a mold 300, liquid paraffin may be dispensed into the groove 310 through the cassette 200 to embed the tissue sample, and a solidified paraffin block may be obtained by cooling liquid paraffin in the mold 300 and/or the cassette 200.

The device 100 may include a first pressing assembly 110 and a second pressing assembly 150. The first pressing assembly 110 and the second pressing assembly 150 may be used separately or in combination.

FIG. 1 is a perspective view of a device for pressing a cassette according to an embodiment of the present disclosure. As illustrated in FIG. 1, the first pressing assembly 110 may be configured to press the outer frame 210 against the mold 300.

The first pressing assembly 110 may include a first pressing member 111, a first abutment member 113, a first sensor 115, and a first guide member 117. The first abutment member 113 may be configured to abut against the outer frame 210 of the cassette 200, to force the outer frame 210 to be held in the groove 310 of the mold 300. The first sensor 115 may be coupled between the first abutment member 113 and the first pressing member 111 and configured to measure a pressing force on the outer frame 210 of the cassette 200.

A user may push the first pressing member 111, and force the first abutment member 113 via the first sensor 115 to abut against an upper face of the outer frame 210 of the cassette 200. Thus, the outer frame 210 of the cassette 200 may be held in the groove 310 of the mold 300, and the pressing force on the outer frame 210 of the cassette 200 may be detected by the first sensor 115 coupled between the first pressing member 111 and the first abutment member 113.

The first pressing member 111 is detachably mounted to the first guide member 117 and slidable on the first guide member 117 in a vertical direction. Thus, the movement of the first pressing member 111 can be guided by the first guide member 117. In the embodiment illustrated in FIG. 1, the first pressing member 111 defines a first hole 1111 through the first pressing member 111 in the vertical direction, and the first guide member 117 includes a first guide shaft 1171 fitted in the first hole 1111. Thus, the first pressing member 111 can slide along the first guide shaft 1171. In some alternative embodiments, the first guide member 117 may include a guide rail, and the first pressing member 111 may define a guide slot, and the guide rail may be fitted in the guide slot. Thus, the first pressing member 111 can slide along the guide rail. Any suitable connection form between the first pressing member 111 and the first guide member 117 can be employed, as long as the first pressing member 111 can slide along the first guide member 117.

In the embodiment illustrated in FIG. 1, the mold 300 may define a first mounting hole 320 at a side of the groove 310 in a length direction of the groove 310, and the first guide shaft 1171 may be fitted in the first mounting hole 320. For example, the first guide shaft 1171 may be fitted in the first mounting hole 320 through threaded connection.

In the embodiment illustrated in FIG. 1, the first guide member 117 may include two first guide shafts 1171, and the two first guide shafts 1171 may be detachably mounted on the mold 300. The first pressing member 111 may define two first holes 1111 through the first pressing member 111 in the vertical direction for passage of the two first guide shafts 1171, respectively. Thus, the first pressing member 111 may be fitted over the first guide shafts 1171 through the two first holes 1111, and the first pressing member 111 may be moved along the first guide shafts 1171 more stably.

In the embodiment illustrated in FIG. 1, the mold 300 may define two first mounting holes 320 arranged at two sides of the groove 310 in the length direction of the groove 310, and the two first guide shafts 1171 may be fitted in the two first mounting holes 320, respectively.

In some embodiments, the number of the first guide shafts 1171 may be three or more, the number of the first holes 1111 in the first pressing member 111 may also be three or more, and the number of the first mounting holes 320 may also be three or more, which is not specifically limited herein.

In the embodiment illustrated in FIG. 1, the first guide shaft 1171 is cylindrical, and the first hole 1111 is circular. In other embodiments, the first guide shaft 1171 may be prismatic, and the first hole 1111 may be polygonal and match the first guide shaft 1171, which is not specifically limited herein.

The first pressing assembly 110 may further include a first elastic member 119 coupled between the first pressing member 111 and the mold 300 and configured to force the first pressing member 111 towards the outer frame 210 of the cassette 200. Thus, the first abutment member 113 can be abutted against the upper face of the outer frame 210 of the cassette 200 under the action of the first elastic member 119. In the embodiment illustrated in FIG. 1, the first elastic member 119 includes a first spiral spring 1191 connected between the first pressing member 111 and the mold 300. In the present embodiment, the first elastic member 119 may include two first spiral springs 1191. In other embodiments, the number of the first spiral springs 1191 may be three or more, which is not specifically limited herein.

The first pressing assembly 110 may include a rod 121 configured to be detachably mounted on the mold 300, the first pressing member 111 is slidably mounted to the rod 121, a free end of the rod 121 extends beyond a surface of the first pressing member 111 away from the mold 300, and the first elastic member 119 is coupled between the free end of the rod 121 and the surface of the first pressing member 111 away from the mold 300. Thus, the first elastic member 119 is coupled to the mold 300 by the rod 121. In the embodiment illustrated in FIG. 1, the first spiral spring 1191 is fitted over the rod 121 and connected between the free end of the rod 121 and the upper face of the first pressing member 111. Thus, the expansion and retraction of first spiral spring 1191 may be guided by the rod 121. The mold 300 may define a second mounting hole 330 at a side of the groove 310 in the length direction of the groove 310, the second mounting hole 330 is located closer to the groove 310 than the first mounting hole 320, and the rod 121 may be fitted in the second mounting hole 330. For example, the rod 121 may be fitted in the second mounting hole 330 through threaded connection.

In the present embodiment, the first pressing assembly 110 may include two rods 121, and the two first spiral springs 1191 are fitted over the two rods 121 and connected between free ends of the two rods 121 and the upper face of the first pressing member 111. The mold 300 may define two second mounting holes 330 arranged at two sides of the groove 310 in the length direction of the groove 310, and the two rods 121 may be fitted in the two second mounting holes 330, respectively. The two first mounting holes 320 and the two second mounting holes 330 may be aligned in the length direction of the groove 310. In other embodiments, the number of the rods 121 may be three or more, and the number of the second mounting holes 330 may be three or more, which is not specifically limited herein.

The first pressing member 111 may define a second hole 1113 through the first pressing member 111 in the vertical direction, and the rod 121 may be fitted in the second hole 1113. Thus, the rod 121 will not interfere with the movement of the first pressing member 111 along the first guide member 117. In the embodiment illustrated in FIG. 1, the first pressing member 111 may define two second holes 1113 for passage the two rods 121, respectively. The two rods 121 may pass through the two second holes 1113 respectively, and the two first spiral springs 1191 may be fitted over the two rods 121 respectively. In other embodiments, the number of the second holes 1113 may be three or more, which is not specifically limited herein.

In the embodiment illustrated in FIG. 1, the rod 121 is cylindrical, and the second hole 1113 is circular. In other embodiments, the rod 121 may be prismatic, and the second hole 1113 may be polygonal and match the rod 121, which is not specifically limited herein.

In some alternative embodiments, the first guide shafts 1171 may be omitted, since the rods 121 may also have guidance effect on the first pressing member 111.

The first pressing assembly 110 may include an adjustment screw 123 screwed to the free end of the rod 121, and the first elastic member 119 is coupled between the adjustment screw 123 and the surface of the pressing member away from the mold 300. In the embodiment illustrated in FIG. 1, the first spiral spring 1191 is coupled between a lower face of the adjustment screw 123 and the upper face of the first pressing member 111. Thus, the pressing force on the outer frame 210 of the cassette 200 may be adjusted by turning the adjustment screw 123 up and down. It could be understood that when the adjustment screws 123 are turned down, the first spiral springs 1191 will be further compressed, and the spring forces pushing the first pressing member 111 towards the outer frame 210 of the cassette 200 increase; and when the adjustment screws 123 are turned up, the first spiral springs 1191 will be less compressed, and the spring forces pushing the first pressing member 111 towards the outer frame 210 of the cassette 200 decrease.

In the present embodiment, the first pressing assembly 110 may include two adjustment screws 123 screwed to free ends of the two rods 121 respectively. The two first spiral springs 1191 are coupled between the first pressing member 111 and the two adjustment screws 123. In other embodiments, the number of the adjustment screws 123 may be three or more, which is not specifically limited herein.

In some alternative embodiments, the adjustment screws 123 may be screwed to the rods 121 close to fixed ends of the rods 121, and the first spiral springs 1191 may be connected between the two adjustments screws and the lower face of the first pressing member 111. Thus, the first pressing member 111 may be pulled towards the outer frame 210 of the cassette 200 by the two tension spiral springs. It could be understood that when the adjustment screws 123 are turned down, the first spiral springs 1191 will be further tensioned, and the spring forces puling the first pressing member 111 towards the outer frame 210 of the cassette 200 increase; and when the adjustment screws 123 are turned up, the first spiral springs 1191 will be less tensioned, and the spring forces pulling the first pressing member 111 towards the outer frame 210 of the cassette 200 decrease.

In the embodiment illustrated in FIG. 1, the first pressing member 111 may have a rectangular-plate shape. The two first holes 1111 are defined close to two ends of the first pressing member 111 in a length direction of the first pressing member 111, respectively, and the two second holes 1113 are defined close to two ends of the first pressing member 111 in a length direction of the first pressing member 111 and located at inner sides of the first holes 1111, respectively. The two first holes 1111 and the two second holes 1113 may be aligned in the length direction of the first pressing member 111.

The first abutment member 113 may include a protrusion 1131 extending towards the outer frame 210 of the cassette 200 in the vertical direction. Thus, the protrusion 1131 of the first abutment member 113 can be abutted against the outer frame 210 of the cassette 200. In the embodiment illustrated in FIG. 1, the first abutment member 113 may have a rectangular-plate shape. The shape of the first abutment member 113 may match a shape of the outer frame 210 of the cassette 200. The first abutment member 113 may include four protrusions 1131 extending towards the outer frame 210 of the cassette 200 at four corners of the first abutment member 113, respectively. Thus, when the first abutment member 113 is abutted against the outer frame 210 of the cassette 200, the four protrusions 1131 touch the upper face of the outer frame 210 of the cassette 200, and four gaps 1133 may be defined between the first abutment member 113 and the outer frame 210 for easy observation of paraffin dispensing from different angles during the embedding process.

The first abutment member 113 may define a first recess 1135 in a side of the first abutment member 113, and the first recess 1135 is configured to position a dispensing tube of a paraffin dispensing device. In the embodiment illustrated in FIG. 1, two first recesses 1135 are defined in two opposite sides of the first abutment member 113 in the width direction of the first abutment member 113. In other embodiments, the first abutment member 113 may define four or more first recesses 1135 in the sides of the abutment member, which is not specifically limited herein.

In the embodiment illustrated in FIG. 1, the first sensor 115 may have a cylindrical shape. An upper face of the first sensor 115 may be mounted to a center of the lower face of the first pressing member 111, and a lower face of the first sensor 115 may be connected to a center of the upper face of the first abutment member 113. The first sensor 115 may be a force transducer such as a piezoelectric force transducer, a vibrating wire force transducer, an inductive force transducer. The pressing force on the outer frame 210 of the cassette 200 can be detected by the first sensor 115, and a signal indicating the value of the pressing force may be sent from the first sensor 115 to a monitor for display to the user.

FIG. 2 is a perspective view of a device for pressing a cassette according to another embodiment of the present disclosure. As illustrated in FIG. 2, the second pressing assembly 150 may be configured to press the inner frame 220 against the mold 300, during the embedding process.

The second pressing assembly 150 may include a second pressing member 151, a second abutment member 153, a second sensor 155, and a second guide member 157. The second abutment member 153 may be configured to abut against the inner frame 220 of the cassette 200, to force the inner frame 220 to be held in the groove 310 of the mold 300. The second sensor 155 may be coupled between the second abutment member 153 and the second pressing member 151, and configured to measure a pressing force on the inner frame 220 of the cassette 200.

The user may push the second pressing member 151, and force the second abutment member 153 via the second sensor 155 to abut against an upper face of the inner frame 220 of the cassette 200. Thus, the inner frame 220 of the cassette 200 may be held in the groove 310 of the mold 300, and the pressing force on the inner frame 220 of the cassette 200 may be detected by the second sensor 155 coupled between the second pressing member 151 and the second abutment member 153.

The second pressing member 151 is detachably mounted to the second guide member 157 and slidable on the second guide member 157 in the vertical direction. Thus, the movement of the second pressing member 151 can be guided by the second guide member 157. In the embodiment illustrated in FIG. 2, the second pressing member 151 may define a third hole 1511 through the second pressing member 151 in the vertical direction, and the second guide member 157 includes a second guide shaft 1571 fitted in the third hole 1511. Thus, the second pressing member 151 can slide along the second guide shaft 1571. The mold 300 may define a third mounting hole 340 at a side of the groove 310 in a width direction of the groove 310, and the second guide shaft 1571 may be fitted in the third mounting hole 340, e.g., through threaded connection.

In some alternative embodiments, the second guide member 157 may include a guide rail, and the second pressing member 151 may define a guide slot, and the guide rail may be fitted in the guide slot. Thus, the second pressing member 151 can slide along the guide rail. Any suitable connection form between the second pressing member 151 and the second guide member 157 can be employed, as long as the second pressing member 151 can slide along the second guide member 157.

In the embodiment illustrated in FIG. 2, the second pressing assembly 150 may include two second guide shafts 1571, and the two second guide shafts 1571 may be detachably mounted on the mold 300. The second pressing member 151 may define two third holes 1511 through the second pressing member 151 in the vertical direction for passage of the two second guide shafts 1571, respectively. Thus, the second pressing member 151 may be fitted over the second guide shafts 1571 through the two third holes 1511, and the second pressing member 151 may be moved along the second guide shafts 1571 more stably. The mold 300 may define two third mounting holes 340 at two sides of the groove 310 in the width direction of the groove 310, and the two second guide shafts 1571 may be fitted in the two third mounting holes 340, respectively. In some embodiments, the number of the second guide shafts 1571 may be three or more, the number of the third holes 1511 in the second pressing member 151 may also be three or more, and the number of the third mounting holes 340 may also be three or more, which is not specifically limited herein.

In the embodiment illustrated in FIG. 2, the second guide shaft 1571 is cylindrical, and the third hole 1511 is circular. In other embodiments, the second guide shaft 1571 may be prismatic, and the third hole 1511 may be polygonal and match the second guide shaft 1571, which is not specifically limited herein.

The second pressing assembly 150 may further include a second elastic member 159 coupled between the second pressing member 151 and the mold 300 and configured to force the second pressing member 151 away from the inner frame 220 of the cassette 200. Thus, the second abutment member 153 can be separated from the upper face of the inner frame 220 of the cassette 200 under the action of the second elastic member 159. Thus, the second pressing assembly 150 may be used to press the cassette 200 containing a tissue sample having different heights to a predetermined position and meanwhile the retraction function of the second elastic member 159 can avoid damage to the tissue sample.

The second elastic member 159 is coupled between the mold 300 and a surface of the second pressing member 151 facing the mold 300. In the embodiment illustrated in FIG. 2, the second elastic member 159 includes a second spiral spring 1591 connected between a lower face of the second pressing member 151 and an upper face of the mold 300. In the present embodiment, the second pressing assembly 150 may include two second spiral springs 1591. In other embodiments, the number of the second spiral springs 1591 may be three or more, which is not specifically limited herein.

In the embodiment illustrated in FIG. 2, and the two second spiral springs 1591 may be fitted over the two second guide shafts 1571, respectively. Thus, expansion and retraction of the second spiral springs 1591 can be guided by the second guide shaft 1571. In alternative embodiments, the second pressing assembly 150 may include a further rod mounted to the mold 300 which may be similar to the rod 121, and the second spiral spring 1591 may be fitted over the further rod.

The second pressing assembly 150 may further include an adjustment ring 161 fitted over the second guide member 157 and arranged between the second elastic member 159 and the mold 300. Thus, the spring force of the second spiral spring 1591 can be adjusted. In the embodiment illustrated in FIG. 2, the second pressing assembly 150 may include two adjustment rings 161 fitted over each second guide shaft 1571 and placed on the upper face of the mold 300. The two second spiral springs 1591 are coupled between the lower face of the pressing member and the upper face of the two adjustment rings 161. Thus, the second pressing member 151 may be pushed away from the inner frame 220 of the cassette 200 under spring forces of the two second spiral springs 1591, and the spring forces of the two second spiral springs 1591 may be adjusted by adjusting the number of the adjustment rings 161. It could be understood that when the number the adjustment rings 161 is reduced, the second spiral springs 1591 will be less compressed, and the spring forces pushing the second pressing member 151 away from the inner frame 220 of the cassette 200 decrease; and when the number of the adjustment rings 161 is increased, the second spiral springs 1591 will be further compressed, and the spring forces pushing the second pressing member 151 away from the inner frame 220 of the cassette 200 increase. The adjustment rings 161 may have the same specification or different specifications.

In some alternative embodiments, the second spiral springs 1591 may be connected between the upper face of the second pressing member 151 and the free end of the second guide shafts 1571. Thus, the second pressing member 151 may be pulled away from the inner frame 220 of the cassette 200 by the two tension spiral springs.

In the embodiment illustrated in FIG. 2, the second pressing member 151 may have a rectangular-plate shape. The two third holes 1511 are defined close to two ends of the second pressing member 151 in a length direction of the second pressing member 151, respectively.

The second abutment member 153 may include a leg 1531 extending towards the inner frame 220 of the cassette 200 in the vertical direction. Thus, the leg 1531 of the second abutment member 153 can be abutted against the inner frame 220 of the cassette 200. In the embodiment illustrated in FIG. 2, the second abutment member 153 may have a rectangular-plate shape. The shape of the second abutment member 153 may match a shape of the inner frame 220 of the cassette 200. The second abutment member 153 may include four legs 1531 extending towards the inner frame 220 of the cassette 200 at four corners of the second abutment member 153, respectively. Thus, when the second abutment member 153 is abutted against the inner frame 220 of the cassette 200, the four legs 1531 touch the upper face of the inner frame 220 of the cassette 200, and four clearances 1533 may be defined between the second abutment member 153 and the inner frame 220 for easy observation of paraffin dispensing from different angles during the embedding process. The four legs 1531 may abut against four corners of the inner frame 220 of the cassette 200, respectively.

The second abutment member 153 may define a second recess 1535 in a side of the second abutment member 153, and the second recess 1535 may be configured to position a dispensing tube of a paraffin dispensing device. In the embodiment illustrated in FIG. 2, two second recesses 1535 are defined in two opposite sides of the second abutment member 153 in a length direction of the second abutment member 153.

The second pressing member 151 may further define a third recess 1513 in a side of the second pressing member 151, and the third recess 1513 and the second recess 1535 are aligned in vertical direction for passage of the second abutment member 153 of the second pressing assembly 150. In the embodiment illustrated in FIG. 2, two third recesses 1513 are defined in two opposite sides of the second pressing member 151 in a width direction of the second pressing member 151.

The second sensor 155 may have a cylindrical shape. An upper face of the second sensor 155 may be mounted to a center of the lower face of the second pressing member 151, and a lower face of the second sensor 155 may be connected to a center of the upper face of the second abutment member 153. The second sensor 155 may be a force transducer such as a piezoelectric force transducer, a vibrating wire force transducer, an inductive force transducer. The pressing force on the inner frame 220 of the cassette 200 can be detected by the second sensor 151, and a signal indicating the value of the pressing force may be sent from the second sensor 151 to the monitor for display to the user.

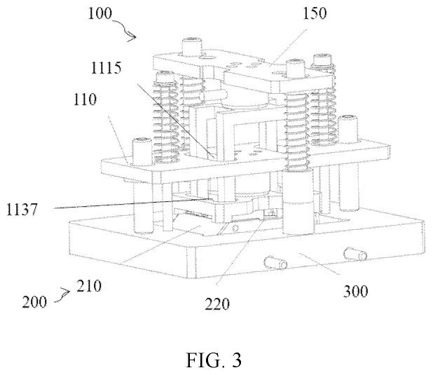

FIG. 3 is a perspective view of a device for pressing a cassette according to yet another embodiment of the present disclosure. As illustrated in FIG. 3, the first pressing assembly 110 and the second pressing assembly 150 may be used in combination.

The first abutment member 113 defines a first slot 1137 through the first abutment member 113 in the vertical direction, the first pressing member 111 defines a second slot 1115 through the first pressing member 111 in the vertical direction, and the first slot 1137 and the second slot 1115 are aligned in the vertical direction for passage of the second abutment member 153. In the embodiment illustrated in FIG. 3, two first slots 1137 are defined in the first abutment member 113 and located at two sides of the first sensor 115 in a length direction of the first abutment member 113; and two second slots 1115 are defined in the first pressing member 111 and located at two sides of the first sensor 115 in a length direction of the first pressing member 111.

The two first guide shafts 1171 and the two rods 121 may be mounted on the mold 300, and the first pressing member 111 is fitted over the two first guide shafts 1171 and the two rods 121 via the two first holes 1111 and the two second holes 1113. Then, the two first spiral springs 1191 may be fitted over the two rods 121, respectively, and the two adjustment screws 123 are screwed to the free ends of the rods 121, respectively. The first ends of the first spiral springs 1191 are placed on the upper face of the first pressing member 111, and the second ends of the first spiral springs 1191 are abutted against lower faces of the adjustment screws 123, respectively.

The two second guide shafts 1571 may be mounted on the mold 300. Four adjustment rings 161 may evenly distributed to the two guide shafts, and the two second spiral springs 1591 may be fitted over the two second guide shafts 1571. The second pressing member 151 may be fitted over the two second guide shafts 1571 via the two third holes 1511. The lower ends of the second spiral springs 1591 are placed on the upper faces of the upmost adjustment rings 161, and the upper ends of the second spiral springs 1591 are abutted against the lower face of the second pressing member 151. The four legs 1531 of the second abutment member 153 pass through the two second slots 1115 in the first pressing member 111, and the two first slots 1137 in the first abutment member 113, sequentially. The third recesses 1513 in the second pressing member 151, the second recesses 1535 in the second abutment member 153, the second slots 1115 in the first pressing member 111 and the two first slots 1137 in the first abutment member 113 are aligned in the vertical direction for passage of the dispensing tube from the paraffin dispensing device.

During the embedding process, the user can turn the adjustment screws 123 manually or automatically to adjust spring forces of the first spiral springs 1191, to push the first pressing member 111 towards the outer frame 210 of the cassette 200, thereby holding the outer frame 210 in the groove 310 of the mold 300 stably. Meanwhile, the pressing force on the outer frame 210 of the cassette 200 can be detected by the first sensor 115, and a signal indicating the value of the pressing force may be sent from the first sensor 115 to the monitor for display to the user. The user can also push the second pressing member 151 manually or automatically towards the inner frame 220 of the cassette 200, thereby holding the inner frame 220 in the groove 310 of the mold 300 stably. Meanwhile, the pressing force on the inner frame 220 of the cassette 200 can be detected by the second sensor 155, and a signal indicating the value of the pressing force may be sent from the second sensor 155 to the monitor for display to the user.

In the device 100 according to embodiments of the present disclosure, the outer frame 210 of the cassette 200 is held in the groove 310 of the mold 300 by the first pressing assembly 110, and the inner frame 220 of the cassette 200 is held in the groove 310 of the mold 300 by the second pressing assembly 150, to improve stability of the cassette 200; furthermore, the pressing force on the outer frame 210 of the cassette 200 may be measured by the first sensor 115 of the first pressing assembly 110, and the pressing force on the inner frame 220 of the cassette 200 may be measured by the second sensor 155 of the second pressing assembly 150, thus the pressing forces can be monitored and adjusted in real time, to improve the quality of the paraffin block during the embedding process.

Embodiments of the present disclosure also provide an assembly including a device 100 according to any of the above embodiments, a cassette 200, and a mold 300. The device 100 may press the cassette 200 to be held in a groove 310 of the mold 300 and measure a pressing force on the cassette 200.

Embodiments of the present disclosure further provide an embedder for automatic embedding tissue samples in paraffin including a device 100 according to any of the above embodiments.

It should be noted that, although the present disclosure has been described with reference to the embodiments, it will be appreciated by those skilled in the art that the disclosure includes other examples that occur to those skilled in the art to execute the disclosure. Therefore, the present disclosure is not limited to the embodiments.

### REFERENCE LIST

100 device
110 first pressing assembly
111 first pressing member
1111 first hole
1113 second hole
1115 second slot
113 first abutment member
1131 protrusion
1133 gap
1135 first recess
1137 first slot
115 first sensor
117 first guide member
1171 first guide shaft
119 first elastic member
1191 first spiral spring
121 rod
123 adjustment screw
150 second pressing assembly
151 second pressing member
1511 third hole
1513 third recess
153 second abutment member
1531 leg
1533 clearance
1535 second recess
155 second sensor
157 second guide member
1571 second guide shaft
159 second elastic member
1591 second spiral spring
161 adjustment ring
200 cassette
210 outer frame
220 inner frame
300 mold
310 groove
320 first mounting hole
330 second mounting hole
340 third mounting hole

1. A device for pressing a cassette, the cassette comprising an outer frame and an inner frame mounted in the outer frame, the cassette being configured to be held in a groove of a mold, and the device comprising:
a pressing member;
an abutment member configured to abut against the cassette;
a sensor coupled between the pressing member and the abutment member and configured to measure a pressing force on the cassette; and
a guide member configured to be mounted on the mold,
wherein the pressing member is detachably mounted to the guide member and slidable on the guide member in a vertical direction.

2. The device according to claim 1, wherein the pressing member defines a first hole through the pressing member in the vertical direction, and the guide member is fitted in the first hole.

3. The device according to claim 1, further comprising:
an elastic member coupled between the pressing member and the mold and configured to force the pressing member towards the outer frame of the cassette, wherein the abutment member configured to abut against the outer frame of the cassette.

4. The device according to claim 3, wherein:
the abutment member comprises a protrusion extending towards the outer frame of the cassette in the vertical direction; and/or
the abutment member defines a first recess in a side of the abutment member, and the first recess is configured to position a dispensing tube of a paraffin dispensing device.

5. (canceled)

6. The device according to claim 3, further comprising:
a rod configured to be detachably mounted on the mold, wherein the pressing member is slidably mounted to the rod, a free end of the rod extends beyond a surface of the pressing member away from the mold, and the elastic member is coupled between the free end of the rod and the surface of the pressing member away from the mold.

7. The device according to claim 6, wherein the pressing member defines a second hole through the pressing member in the vertical direction, and the rod is fitted in the second hole.

8. The device according to claim 6, further comprising:
an adjustment screw screwed to the free end of the rod, wherein the elastic member is coupled between the adjustment screw and the surface of the pressing member away from the mold.

9. The device according to claim 1, further comprising:
an elastic member coupled between the pressing member and the mold and configured to force the pressing member away from the inner frame of the cassette, wherein the abutment member is configured to abut against the inner frame of the cassette.

10. The device according to claim 9, wherein the elastic member is coupled between the mold and a surface of the pressing member facing the mold.

11. The device according to claim 9, further comprising:
an adjustment ring fitted over the guide member and arranged between the elastic member and the mold.

12. The device according to claim 1, wherein the sensor is elastically coupled to a monitor, and is configured to send a signal to the monitor for display of a measured value of the pressing force on the cassette.

13. The device according to claim 3, further comprising:
a further pressing member;
a further abutment member configured to abut against the inner frame of the cassette;
a further sensor coupled between the further pressing member and the further abutment member and configured to measure a pressing force on the inner frame of the cassette, and
a further guide member configured to be mounted on the mold,
wherein the further pressing member is detachably mounted to the further guide member and slidable on the further guide member in the vertical direction.

14. The device according to claim 13, wherein the further pressing member defines a third hole through the further pressing member in the vertical direction, and the further guide member is fitted in the third hole.

15. The device according to claim 13, wherein the abutment member defines a first slot through the abutment member in the vertical direction, the pressing member defines a second slot through the pressing member in the vertical direction, and the first slot and the second slot are aligned in the vertical direction for passage of the further abutment member.

16. The device according to claim 13, wherein the further abutment member comprises a leg extending towards the inner frame of the cassette in the vertical direction.

17. The device according to claim 13, further comprising:
a further elastic member coupled between the further pressing member and the mold and configured to force the pressing member away from the inner frame of the cassette.

18. The device according to claim 17, wherein the further elastic member is coupled between the mold and a surface of the further pressing member facing the mold.

19. The device according to claim 17, further comprising:
an adjustment ring fitted over the further guide member and coupled between the further elastic member and the mold.

20. The device according to claim 13, wherein the further abutment member defines a second recess in a side of the further abutment member, the further pressing member defines a third recess in a side of the further pressing member, and the second recess and the third recess are aligned in the vertical direction to position a dispensing tube of a paraffin dispensing device.

21. An embedder for automatically embedding tissue samples in paraffin with an integrated device according to claim 1.
